(19) United States
(12) Patent Application Publication
(10) Pub. No.: US 2015/0165229 A1
(43) Pub. Date: Jun. 18, 2015

(54) PHOTOTHERAPY TREATMENT DEVICE

(71) Applicant: Djalma Luiz RODRIGUES, Sao Paulo (BR)

(72) Inventor: Djalma Luiz RODRIGUES, Sao Paulo (BR)

(21) Appl. No.: 14/573,159

(22) Filed: Dec. 17, 2014

(30) Foreign Application Priority Data
Dec. 17, 2013 (BR) 1020130324892

(51) Int. Cl. A61N 5/06

(52) U.S. Cl. CPC A61N 5/0621; A61N 2005/0651; A61N 2005/0628; A61N 2005/0632

(57) ABSTRACT

The present invention relates to a hospital item of equipment for application to phototherapy treatments comprising a body (9) provided with at least one source of phototherapy light (5) in the form of at least one high-intensity LED capable of emitting suitable irradiance suitable for phototherapy treatment, at least one internal processing element such as a microprocessor and one or more collimating lenses (3) positioned adjacent to said at least one high-intensity LED (5) in the direction of brightness/irradiation emitted, at least one focus varying mechanism (2, 4) being further provided, which is operatively associated to one or more lenses (3), a radiometer associated with or without the use of wire with rechargeable battery being further provided, having a connector in the form of an electromagnetic-wave-transmitting antenna, in the Wi-Fi, Bluetooth, Zigbee or any other standard.

This application claims the benefit of Brazilian Patent Application Serial No. BR102013032489-2, filed Dec. 17, 2013, which is hereby incorporated by reference in its entirety.

### FIELD

The present invention relates to a piece of hospital equipment for application of phototherapeutic treatments, capable of providing uniform, homogeneous and varying distribution of irradiance over the body of a newly born patient (new-born baby) regardless of the new-born baby's body area, with a view to maximize the efficacy of the treatment and/or reduce side effects thereof.

The present invention also relates to hospital equipment for application in phototherapeutic treatments, which, in addition, enable specific adjustments of irradiance in a controlled and automatized manner, without the need for manual intervention by an operator.

### BACKGROUND

Hyperbiluribinemia is an anomaly, usually observed in new-born babies, related to an increase in levels of serum bilirubin in their organism, which may cause the appearance of a yellowish color in the sclerae, mucous membranes and/or skin, called icterus. Neonatal icterus affects about 60% to 80% of newborns and requires adequate treatment.

The treatment of hyperbilurubinemia began in England in the late 1950's, when it was observed that sunlight emitted onto the skin of an icteric newborn would reduce remarkably the serum bilirubin levels. In the face of this finding, the first phitotherapy apparatus with fluorescent lamps were developed, which act on the light spectrum visible in the blue range (light wavelength ranging from 400 to 550 nm), which are used until today. These phototherapy apparatus are capable of transforming the bilirubin molecules into non-toxic isomers that are soluble in water, to be eliminated from the organism by means of the kidneys, through sudoresis (diaphoresis), through the pores, etc.

With the advance of the technology over the years, other types of light sources were developed which exhibit greater efficiency, such as fluorescent lamps, capable of emitting light in the blue spectrum, halogen lamps, LED lamps and, at present, high-intensity LEDs, which are used, for instance, on the Bilitron® 3006 phototherapy equipment produced by Fanem® Ltda. The high-intensity LEDs are more economical, efficient and consume less energy than conventional LEDs, by virtue of their preferred composition of indium and gallium nitride (conventional LEDs comprise only gallium nitride).

In spite of the high efficiency and reliability of the LEDS, one of the drawbacks observed in the use thereof in phototherapy is that they do not exhibit uniformity and homogeneity of radiation on the treatment surface of a patient's bed, due to the lack of more efficient optics, since they consists of punctual light sources. It should be noted that uniform and homogeneous distribution of irradiance on the phototherapeutic treatment surface represents a significant improvement in the efficacy of phototherapeutic treatments.

Particularly, the LEDs used in phototherapeutic treatments may be classified into two categories according to the level of optical power generated: low light power conventional LEDs (less than 50 mW radiation in the range from 400 to 550 nm); and high-brightness LEDS (more than 500 mW radiation in the range 400 to 550 nm).

With regard to conventional LEDS, which exhibit low optic power, they are usually configured in the form of a continuous row without spacing between them, in order to increase the radiation intensity on the treatment surface of the patient's bed. As a result, it becomes necessary to implement hundreds of conventional LEDs in order to generate acceptable radiation on a treatment surface. It should be noted that, due to the difference in light intensity (visible irradiance power emitted by a light source available in a given direction) usually existing between the LEDs, on observes that there is ideal uniformity and homogeneity of the distribution of irradiance on the treatment surface. For this reason, each part of the patient's body may receive a different dose of irradiance (higher or lower than ideal), chiefly when he is offset with respect to an ideal position on the bed, which may impair the efficacy of the phototherapeutic treatment, causing the spot irradiated onto the patient's body not to be uniform. The ideal thing is to guarantee a uniform treatment area in order to ensure that all the body areas of the patient will receive the adequate therapeutic irradiance dose.

On the other hand, the use of high-brightness LEDs, which has high radiation, enables a considerable reduction of the number of LEDs employed for the order of tens of units. Thus, high-brightness LEDs may be positioned at longer distances between them as compared to conventional LEDs, when they are associated to an efficient and differentiated optical system, this may result in a great difference in the treatment. However, as in happens in the case of conventional LEDs, one does not observe uniformity or homogeneity of the distribution of irradiance on the treatment surface, as explained before.

Further, in order for the phototherapy to be carried out in an effective manner, it is recommendable to monitor and control constantly the intensity and distribution of light to be applied to the patient's body.

Usually, such monitoring of the intensity of light is made manually by an operator, who uses a device capable of capturing the light energy emitted by the light source, as for example, a dedicated radiometer that measures irradiance (amount of light or energy emitted per unit of area in a determined range of the spectrum). More specifically, a radiometer is capable of providing irradiance values in a determined range of the light spectrum of a light source.

In phototherapeutic applications, the radiometer should be configured so as to measure irradiance in the visible blue spectrum, usually employed in phototherapy for the treatment of hyperbilirubinemia, since it is more effective in transforming bilirubin molecules to isomers.

Thus, from the measurement made by the radiometer, the operator (a doctor, male nurse or technician) adjusts (increases or decreases) the intensity of the light source of the phototherapy equipment according to the most different clinical protocols. In other words, the control of light intensity of the light source of the phototherapy equipment is also made manually by the operator.

In this way, both the monitoring of radiance and the control of intensity of the light source are usually made manually, which makes it difficult to obtain a precise and stable adjustment in the irradiance received by the patient, besides increasing the risk of susceptibility to human failures (for instance: wrong or late decision making on the part of the operator), and further it is necessary to dedicate a specific operator for carrying out these tasks.

In this regard, it is important to note also that, if the light sources emit a larger amount of irradiance than necessary, the useful life of the equipment is reduced. On the other hand, if the light source emits less irradiance than necessary, the efficacy/performance of the phototherapy may be impaired (aggravated when the patient is offset with respect to an ideal treatment position).

In the event that the phototherapy is not carried out or is made with insufficient intensity, hyperbilirubinemia may evolve to a more serious condition, namely kernicterus.

This matter is dealt with by the paper “ENCEFALOPATIA BILIRRUBÍNICA (KERNICTERUS)—Aspectos Fisiopatológicos e Clinicos” (BILIRUBINIC ENCEFALOPATHY—Phisyopathologic and Clinical Aspect), by Dr. Paulo R. Margotto, Professor of the medical course of the Escola Superior de Ciências da Saúde (ESCS)/SES/DF, which is hereby incorporated by reference in its entirety, to whom kernicterus results from the toxicity of bilirubin to the cells of the ganglions of the base and various nuclei of the brain trunk or, in other words, a pathologic diagnosis that is characterized by impregnation of brain trunk nuclei with bilirubin and refers to the yellowish coloration of these nuclear areas).

In clinical practice, the work kernicterus is used in interchange with the term bilirubin encephalopathy (the latter refers to the acute manifestations of bilirubin toxicity in the first weeks of life, whereas kernicterus refers to the permanent clinical sequels of bilirubin toxicity).

In the 1950's, with the use of exanguino-transfusion (exchange transfusion: the exchange of a patient's blood) and the appearance of phototherapy equipment, there was a great decrease in kernicterus full-term newborns, and the attention was diverted to pre-term newborns.

In the 1970's, there was a decrease in kernicterus in pre-term newborns as a result of better general care and early and spread use of phototherapies. In extreme pre-term newborns, kernicterus has been reported with low levels of bilirubin (8 mg %). Various other components of the pathogenesis of kernicterus are not related to the serum bilirubin level, like albumin-binding the capacity, acidosis, serum albumin level, use of drugs that compete with bilirubin by albumin, and the duration of contact between free or albumin-bonded bilirubin and the brain endothelium.

According to this article, almost all the children described recently with kernicterus exhibit very high bilirubin levels (higher than 30 mg %). In close-to-full-term newborns, cases of kernicterus with bilirubin level ranging from 5.2 to 14.4 mg % were detected, which indicated that these newborns were very ill.

Describing in greater detail, free bilirubin is toxic to synapses and injures the neurons and the cell organelles. Early neuronal necrosis is followed by cellular loss, gliosis and demyelination in the affected areas. Bilirubin causes neuronal injury in specific areas of the brains with the highest utilization of oxygen, chiefly the pale globe, certain thalamic nuclei, subthalamic nuclei, black substance, hippocampus, hypothalamus, nuclei of the VIII skull pair.

In the case of kernicterus the injuries are more frequent in the globus pallidus (pale globe) (especially at the posteromedial border) and subthalamic nuclei. The involvement of the brain cortex neurons is not a characteristic of kernicterus; when present, it seems to be related primarily with the concomitant hypoxic-ischemic injury.

About 50% of the newborns with kernicterus observed in the autopsy exhibited extraneuronal injuries such as necrosis of kidney tubular cells, intestinal mucosa cells and pancreas cells.

Observing the condition from this point of view, it is also necessary to guarantee, as much as possible, that the patient will receive the phototherapy treatment in the correct intensity, in order for the treatment to be effective. The efficacy of the phototherapy depends upon the light intensity (power of the irradiance emitted by a light source available in a determined direction), upon the wavelength (color) of the light, as well as upn the skin-surface area exposed to the light.

Thus, in order for the monitoring and control of the quality (dose) of light to be applied to a newborn to be possible, there is a need to capture the energy emitted by the light source. Such capture can be made by means of a radiometer, which consists of an apparatus capable of measuring irradiance (amount of light or energy emitted per unit of area in a determined spectrum), that is to say, capable of providing irradiance values in a determined range of the light spectrum of a known source of light. In phototherapeutic applications, the radiometer should be configured to measure irradiance in the visible blue spectrum, usually employed in phototherapy for the treatment of hyperbilirubinemia, because it is more effective in transforming the bilirubin molecules into isomers.

However, light irradiance meters or radiometers/photometers have not evolved at the same velocity as lamps/sources of light. The radiometers that are used at present in the treatment of hyperbiliruminemia consider to be a typical standard of light source only a type of lamp, usually fluorescent lamps, which may entail inexact measurements, if one wishes to measure the irradiance of another type of light source that is the object of patent applications filed by the present applicant, namely: PI 9103778-6, MU 8400812-1, pi 0904575-9 AND pi 1002129-9, which are hereby incorporated by reference herein in their entirety.

For example, in tests and assays made at the Laboratório de Óptica do Instituto de Pesquisas Tecnológicas de São Paulo (IP) (Optics Laboratory of the Technological Research Institute of São Paulo), it was possible to find that each type of lamp exhibits a specific intensity of light for each range of the light spectrum. Therefore, the radiometers known at present are not capable of providing irradiance values with accuracy for the different types of light source, suitable for the treatment of hyperbilirubinemia.

The scientific article “An approach to the management of hyperbilirubinemia in the preterm infant less than 35 weeks of gestation”, by M J Maisels, J F Watchko, V K Bhutani and D K Stevenson, and published in the Journal of Perinatology (2012), which is hereby incorporated by reference herein in its entirety, states properly that, taking as a basis a study made on newborns with less than 35 weeks gestation, a more aggressive phototherapeutic treatment (with greater intensity) causes an increase in the mortality of at least 5%. On the basis of this study, one has estimated that with 89% probability it was the aggressive phototherapy that increased the mortality rate in the subgroup analyzed. In the case of premature newborns with less than 100 grams, the aggressive phototherapy caused an increase of 19% in the mortality.

According to the article, the reasons for this finding are not clear, but the thin and gelatinous skin of these babies causes the light to penetrate deeper into the patient's body, damaging the cell membranes and the DNA. In the studies of the association of neonatal research, the average irradiance level reported was of 22 to 23 mW cm⁻² nm⁻¹, and the level on which the study focused ranged from 15 to 40 mW cm⁻² nm⁻¹.

Thus, it is clear that there is a need to guarantee correct irradiance to be applied to each patient for the treatment of hyperbilirubinemia as a function of his weight, the bilirubin percentage in his blood and as a function of the phototherapy equipment that treats him, so much so in the light of more recent studies that have indicated an increase in mortality in a case of more aggressive phototherapeutic treatments.

Finally, it is necessary that the phototherapeutic equipment achieves the efficient and uniform spreading of luminosity on an area that corresponds to a fair part of the patient's body. It would be useless to apply high irradiance to a part of the patient's body, if in an adjacent area the irradiance value were considerably lower.

Various prior-art items of equipment sought to solve one or more of the above-mentioned problems. However, even though they have managed to solve isolated problems, none of them has really managed to deal with all the variables so as to actually increase the overall efficacy and safety of phototherapy.

The European Patent document EP 0616820, which is hereby incorporated by reference herein in its entirety, describes a system for application in neuronal phototherapy, which comprises a source of light, a hospital bed, a light detector and a regulator capable of adjusting automatically the intensity of the light delivered to the bed on the basis of the measurements made by the light detector.

However, the equipment described in this European Patent document is not capable of enabling specific adjustments of irradiance in different areas or regions of the patient's bed surface, which reduces the efficacy/performance thereof, besides limiting its application field. Additionally, it becomes more difficult to achieve uniform and homogeneous distribution of irradiance throughout the surface of said bed, chiefly in its peripheral portions (borders or ends). Further, this equipment does not enable a compensation for a possible undesired drop in irradiance in some regions of said bed. It is important to observe that the equipment requires the use of optical fibers to transmit the light from the source to the patient and also from the region of contact of the bed with the patient to the light detector (photodetector), since the light source is not directed toward the patient, which raises the production costs.

The Bilitron® 3006 equipment produced by the company Fanem®, described and defined in U.S. Pat. No. 8,202,307, of the same applicant, which is hereby incorporated by reference herein in its entirety, is microprocessed, has five high-intensity LEDs that emit blue light in the desired spectrum and has means for proportional (progressive) control of radiance, based on the radiance value captured by a radiometer that is operatively associated to it.

With regard to the difficulty in guaranteeing efficient and uniform spreading of luminosity over the incubator bed, this undesired situation was inherent, above all in the items of equipment that used LEDs and halogen lamps. Bearing this in mind, the present applicant has developed the medical equipment of Brazilian patent application PI 1002129-9, which is hereby incorporated by reference herein in its entirety. This equipment comprises at least one holographic diffuser positioned between the light source and the bed, which spreads luminosity in a more homogeneous manner throughout the whole body of the patient. Preferably, one used a holographic diffuser film with light transmittance higher than 85%.

Finally, with a view to guarantee the maintenance of a desired level of irradiance for each patient to be treated and his specificities, further taking into account the type of emitting light source, the present applicant has developed the phototherapy equipment and the system for measuring irradiance of Brazilian patent application PI 0904575-9, which is hereby incorporated herein by reference in its entirety. This system is provided with at least one light detecting means, capable of capturing light emitted by the light source, provided with an optical sensor capable of enabling conversion of the impinging light intensity to an electric signal, as for example, electric current or voltage.

The optical sensor is operatively associated to a conditioning electronic circuit, so that the physical magnitude can be converted to an analogical electric signal, and so that it will be amplified and/or converted to a digital electric signal later.

The system also comprises a means for selecting the type of light source capable of enabling selection between a fluorescent lamp, a halogen lamp, a conventional LED (as for example, a gallium nitride one), a high-intensity LED or still a means for emitting light, the intensity of which one wishes to measure, suitable for the treatment of hyperbilirubinemia, since the configuration of the system is flexible and adaptable to any type of lamp. The means for selecting light source consists of a key, an electronic switch or any other adequate means that enables selection of variables.

The system further comprises a control unit (microprocessor, programmable microcontroller or the like), associated operatively to the light detection means and to the means for selecting the type of light source, configured to calculate at least one irradiance value from the data coming from the light detection means and the light-source-type selection means.

More specifically, the control unit is configured to apply at least one correction factor to the irradiance value initially calculated, according to the type of light source selected through the selection means. In this way, the correction is preferably made by means of a computer algorithm to be executed by the microprocessor or microcontroller.

Preferably, the correction can be carried out by multiplying at least one correction factor applied to the irradiance value initially calculated, according to the type of light source selected through the selection means. After many studies and analyses, the applicant calculated a correction factor such that consists, for instance, of the relationship between irradiance achieved experimentally and reference irradiance for each type of light source, the reference irradiance consisting of a theoretical value (for example, obtained from a known standard irradiance value per wavelength) or a specific value for a pre-established or predetermined type of light source or (for example, fluorescent lamp).

In this regard, just as researched and developed by the applicant, one should obtain at least one correction factor for each type of light source, except for the reference source. If necessary, one may implement multiple correction factors for each type of light source, in order to achieve greater measurement accuracy.

Naturally, one may use other magnitudes proportional to the irradiance or still other methods of calculating correction factor. Correction factors may be stored in the internal memory itself of the microcontroller/microprocessor or still be stored in an external memory.

Optionally, the correction may be made by using specific tables for each type of light source, each table being obtained experimentally by measuring the light intensity or some corresponding electric magnitude, such as voltage or electric current as a function of the variation of the wavelength (spectrum) of the light emitted by the respective source of light. These tables may be stored in the internal memory itself of the microcontroller/microprocessor, or still be stored in an external memory.

Moreover, the control unit of the system is also configured to enable adaptation/calibration of the optical sensor for any type of light source, even that were not foreseen initially in design and that may further be developed in the future. For this purpose, one foresees a calibration function in the system for new types of light sources, which should be accessed prior to using the new source of energy.

Therefore, in the face of the foregoing, there is a gap in the prior art, which the present invention aims at filling, as can be seen later in this specification.

### SUMMARY OF THE INVENTION

A first objective of the present invention is to provide an item of hospital equipment for application in phototherapeutic treatments that enables uniform and homogeneous distribution of irradiance over the patient's body, regardless of his position on the hospital bed, so as to provide optimum coverage of irradiance over the patient's body surface.

Besides, it is also a second objective of the present invention to provide an item of hospital equipment for application in phototherapeutic treatments, capable of automatically maintaining the stability and precision of the irradiance received by the patient, so as to prevent unsuitable doses of irradiance from being applied to the patient according to the medical decisions with adequate protocols, without the need for manual interference by an operator at each moment, which optimizes the efficacy of the treatment and further increases the useful life of the devices used.

Additionally, it is a third objective of the present invention to provide an item of hospital equipment for application in phototherapeutic treatments, which enables automatic monitoring and control of the distribution of the irradiance received by a patient, without the need for manual interference of an operator and overcoming the drawback of using an autonomous radiometer.

In addition, it is a fourth objective of the present invention to provide an item of hospital equipment for application in phototherapeutic treatments, which manages to combine accurate and proportional control of radiance with the capability of spreading radiation homogeneously, so that the equipment itself will compensate the increase or decrease in the average irradiance value (achieved by reducing or increasing the light spread area) with the respective proportional increase or decrease in the emitted intensity value, taking into account the calibration values used and preventing the patient from being subjected to an insufficient or excessive (aggressive) dose of irradiance by improving the measurement of irradiance over the spot irradiated, using collimating lenses of opaque standard or the like, and of combination for better homogeneous optical distribution.

Finally, it is a fifth objective of the present invention to provide an item of hospital equipment for application in phototherapeutic treatments, which further comprises at least one connector for an optical sensor and/or a connector for communication with a microcomputer, such as an RS-232 connector, a USB connector or still any other functional connector. The equipment may also have a connector in the form of an antenna for transmitting electromagnetic waves, in the Wi-Fi, Bluetooth, Zigbee standard or any other. In this way, it is possible, for instance, to control a plurality of phototherapy items of equipment in a central room of a maternity hospital, without the need for a nurse to follow the treatment all the time, as well as to connect accessories like a radiometer with or without wire.

The objectives of the present invention are achieved by means of a hospital item of equipment for application in phototherapeutic treatments, which comprises a body provided with at least one source of phototherapeutic light in the form of at least one high-intensity LED capable of emitting irradiance suitable for phototherapeutic treatments, at least one internal processing element with a microprocessor and one or more collimating lenses positioned adjacent to said at least one high-intensity LED in the direction of the brightness/irradiation emitted, being further provided with at least one mechanism for varying the focus operatively, associated to one or more lenses.

### BRIEF DESCRIPTION OF THE DRAWINGS

FIG. 1 illustrates a comparative photograph of the spreading of brightness and irradiance emitted by a phototherapy item of equipment with high-intensity LEDS, comparing it with the phototherapy equipment of the present invention;

FIG. 2 is a schematic figure of the spreading of luminosity and irradiance emitted by a phototherapy item of equipment with high-intensity LEDS;

FIG. 3 illustrates comparatively four patters of the spread of brightness and irradiance on phototherapy apparatus, with two patterns showing bad spreading, a third intermediate pattern, and a fourth pattern showing homogeneous spreading;

FIG. 4 is a schematic figure of the phototherapy equipment of the present invention ion two operation situations;

FIG. 5 is a schematic side view of a first non-limiting variation of the phototherapy equipment of the present invention;

FIG. 6 is a schematic bottom view of a first non-limiting variation of the phototherapy equipment of the present invention;

FIG. 7 is a schematic top view of a first non-limiting variation of the phototherapy equipment of the present invention;

FIG. 8 is a schematic view of a convex lens;

FIG. 9 is a schematic view of a spherical lens;

FIG. 10 is a schematic view of a second non-limiting variation of the phototherapy equipment of the present invention in a first focus situation;

FIG. 11 is a schematic view of a second non-limiting variation of the phototherapy equipment of the present invention in a second focus situation;

FIG. 12 is a schematic view of a third non-limiting variation of the phototherapy equipment of the present invention in a first focus situation;

FIG. 13 is a schematic view of a third non-limiting variation of the phototherapy equipment of the present invention in a second focus situation;

FIG. 14 is a schematic top view of the equipment of the present invention operating in a hybrid incubator with closed dome;

FIG. 15 is a schematic top view of the equipment of the present invention operating on a hybrid incubator with open dome.

### DETAILED DESCRIPTION OF THE INVENTION

The phototherapy equipment 1 of the present invention can assume a number of variations, but it has as a premise the fact that it emits irradiation/brightness in a substantially uniform manner throughout the patient's body, besides enabling easy and rapid variation of the area in which brightness/irradiance is emitted.

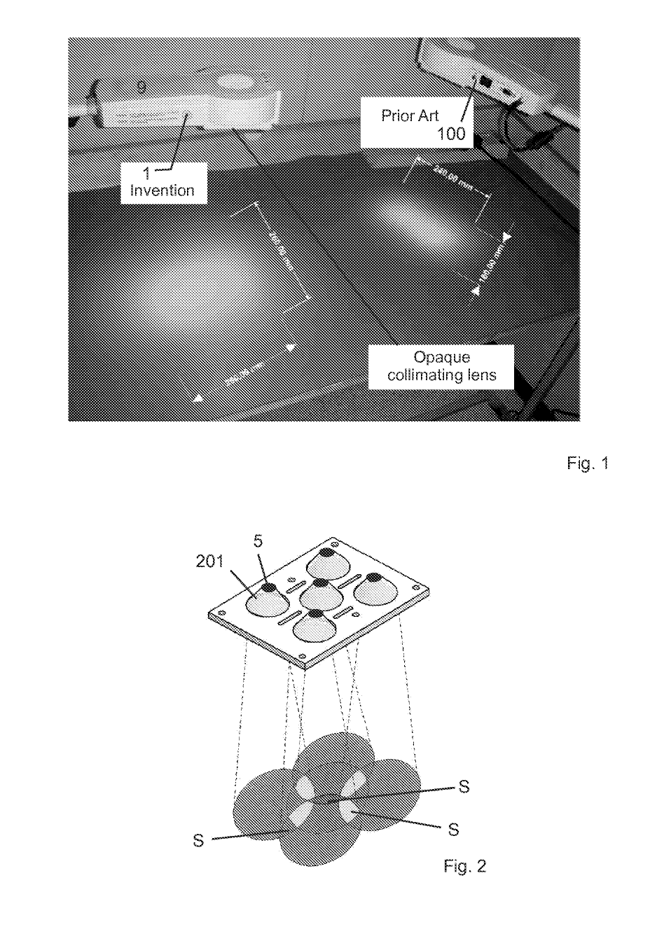

A comparison between the equipment 1 of the present invention and a phototherapy item equipment 100 of the prior art can be found in FIG. 1, clarifying that both use exactly the same source of illumination (the same identical five high-intensity LEDS).

One can see, with the present phototherapy equipment 1, the illuminated useful area exhibits oblong shape of about 260.00 mm×280.00 mm. The prior-art phototherapy apparatus 100, in turn, exhibits an illuminated useful area exhibits oblong shape of 180.00 mm×240.00 mm. Therefore, the advantage of the equipment 1 of the present invention is evident, which results, as will be better explained later, from the use of at least one substantially opaque collimating lens or the like.

It should be emphasized that at present such apparatus are used on pedestals with movable and articulated rods, or even by a flexible system, when the phototherapy equipment is moved away from the patient by means of rods. However, in the case of the conventional pieces of equipment, there is means for correcting the light focus, which makes it difficult to achieve the given irradiance value for the treatment of each patient.

In essence, the phototherapy equipment 1 of the present invention is constituted essentially by a body 9 provided by at least one source of phototherapeutic light of the high-intensity-LED type (preferably, but not compulsorily, five high-intensity LEDs are used) of extremely high light irradiation in the spectral range of blue color, more accurately in the wavelength of 450 nm and which will be commented in greater detail later. Optionally, one further provides at least one ventilator and at least one internal battery (not shown).

All the components are electrically connected to a microprocessor circuit capable of being adjusted by means of control and programming or remote-control actuators (not shown), with their functions being monitored by means of a viewer and a connector for an optical sensor, or digitalized by means of a connection (also not shown), which can be coupled to a microcomputer or the like.

As it is known by those skilled in the art, high-intensity LEDs emit high light power in a small range of the spectrum of electromagnetic radiation (blue), without emission of infrared radiation, which would cause the body to warm up, and also without emission of ultraviolet radiation, which may cause damages to the newborn's skin.

One further provides at least one control panel in the preferred form of a display, which enables correct monitoring and handling of the equipment 1. This display is preferably an alphanumeric liquid-crystal screen (LCD display), which enable reading messages, but may assume other configurations, such as a panel with warning lamps, a small keyboard or still any other functional solution. Evidently, if necessary or desirable, it is possible to devise a piece of equipment 1 according to the teachings of the present invention with a remote display or even without any kind of display.

The equipment 1 further comprises at least one connector for the optical sensor and/or at least one connector for communication with a microcomputer, such as an Rs-232 connector, a USB connector or still any other functional connector. The equipment may also have a connector in the form of an electromagnetic-wave transmitting antenna in the standard Wi-Fi, Bluetooth, Zigbee or still any other. In this way, it is possible, for instance, to control a plurality of phototherapy items of equipment 1 in a central maternity room, without the need for a nurse following the treatment in full time, as well as to connect accessories such as a wire-less radiometer.

The great innovation of the equipment 1 lies in its capability of providing variable spreading of the radiance/brightness in a uniform manner throughout the patient's body, coupled with the capability of varying this area, by virtue of the existence of a focus changing means that will be described later.

In the case of the prior-art equipment 100, each high-intensity LED 5 had a collimator 201 applied to it, the main function of which was to direct all light rays emitted by it. However, as already schematically illustrated in FIG. 2, the efficacy of this system was not ideal, since it ended up overlapping (S) the irradiance/brightness emitted by the high-intensity LEDs 5 in the center of the illuminated area. This type of solution is far from being ideal because, when a patient makes a phototherapy treatment, a part of his body (that which is positioned in the center of the illuminated area) receives a given irradiance value, and the portions of his body adjacent to this area receive much lower irradiance. As a result, even if this patient is receiving a medium irradiance value suitable for his treatment, parts of his body are exposed to excessive radiance and parts are exposed to insufficient irradiance, which may reduce the efficacy of the treatment and, in extreme cases, subject the patient to an aggressive phototherapy.

One should add the fact that the patient moves constantly, thus losing the focus and, as a result, the treatment becomes less effective. On the other hand, in the present invention the adjustment by varying and innovatory focus is independent from the traditional pedestals with rods.

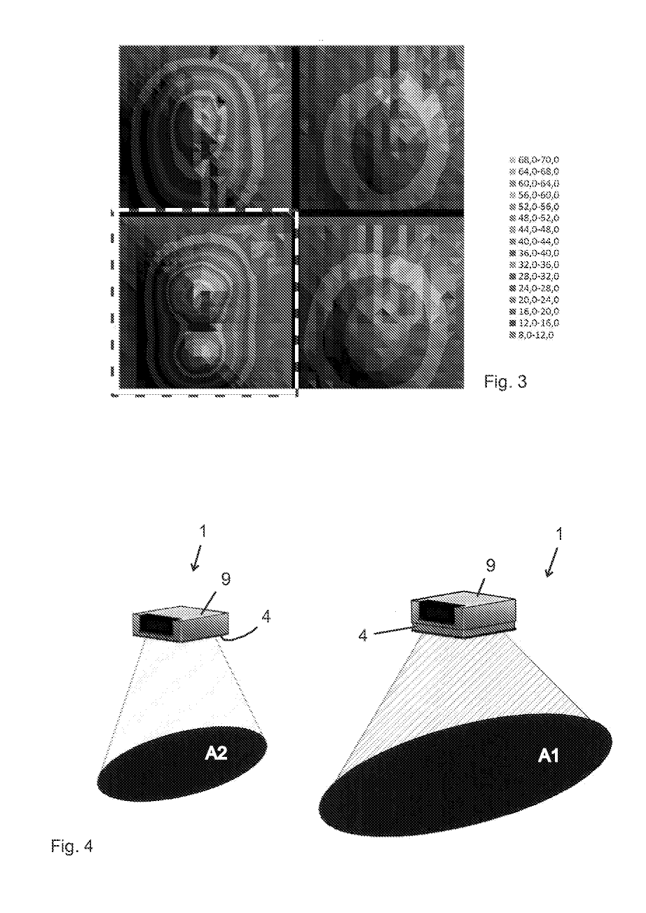

Further in this line, FIG. 3 illustrates different situations of directing the light rays emitted by the high-intensity LEDs 5. The lower left square (highlighted in the figure within a dashed-line rectangle) illustrates a very unfavorable situation. There one can clearly see regions with high concentration of irradiance (which correspond to the two “mountains” or “heights”) flanked by regions with quite lower irradiance, with the dark region between them. This situation corresponds to the spreading of some advanced phototherapeutic items of equipment at present.

The upper left square illustrates another even more unfavorable spreading situation, but it forms a “mountain” or “height”, where the central portion has high concentration of irradiance, which decreases gradually as it moves away from the center. Evidently, parts of the patient's body are subjected to varied irradiance values. It is even intuitive that the treatment is not so effective as if the irradiance and brightness were spread more uniformly.

The lower right square represents a more favorable situation, in the form of a lower “mountain” of broader top, which means that a larger area receives a medium value of higher irradiance, although the central portion still receives quite higher values than the rest of the area.

Finally, the ideal spread situation, which is presented by the equipment 1 of the present invention is that illustrated in the upper right square, having shape similar to that of the small height with a quite broad base. One can see clearly that a fair part of the illuminated area receives a constant medium irradiance value, without great differences between the center (top of the elevation) and the periphery (base of the height). This situation is much more favorable, because it prevents parts of the patient's body from being subjected to an excessive irradiance value, while other adjacent areas receive insufficient irradiance.

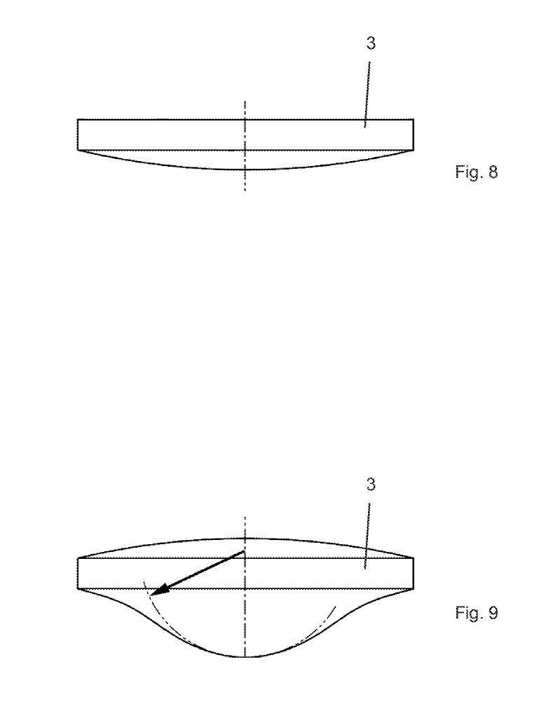

In order to achieve quite homogeneous spreading, the equipment 1 uses one or more opaque collimating lenses 3 positioned adjacent to the high-intensity LEDs 5 in the direction of brightness/irradiation emitted, which provide much greater uniformity with respect to the lenses used at present, which is corroborated by FIGS. 1 and 3. Preferably, one uses a single opaque collimating lens 3, just as illustrated in FIG. 8. However, it is evident that other solutions that achieve equivalent results may be employed, without the resulting equipment failing to be included in the protection scope of the accompanying claims.

Preferably, the lens 3 has a substantially convex shape, defining a first plane surface 30 on which the rays fall, and a second convex surface 300, from which the rays leave the lens.

At least a part of the plane surface and/or the convex surface mat or opaque finish, so as to cause diffraction of the light rays that cross them. This mat or opaque finish may be made by jetting, application of adhesive or any other chemical product. The variation in the position and extent of the jetting depends of the type of light source used, the geometry and the number of lenses employed, among other factors. One may further make opaque the desired surface by making a few patterns or drawings, so as to obtain the Fresnel effect, and the same lens may have different opacities.

Alternatively, one may further use a spherical lens, which is a lens of irregular shape, developed for reducing or eliminating differences in the pattern of light emission of a given emitting body, or non-homogeneity of the source of light (also called spherical aberration). For this purpose, this type of lens, which is more effective, has other shape than the convex or cylindrical one, just as shown in FIG. 9.

Another great difference of the present equipment is the capability of varying the focus adjustment, enabling increase and decrease in the illuminated/irradiate area, which is an innovation that has not been used in phototherapy yet (and often in areas of illumination, microscopy, photography, etc. This adjustment may be made by hand or by means of an electric mechanism, which, coupled with the progressive/proportional control of radiance, make it much more effective in the treatment of the most varied types of patient, in the most varied hiperbiliruminemia intensities, guaranteeing that situations of underexposure or overexposure will be prevented.

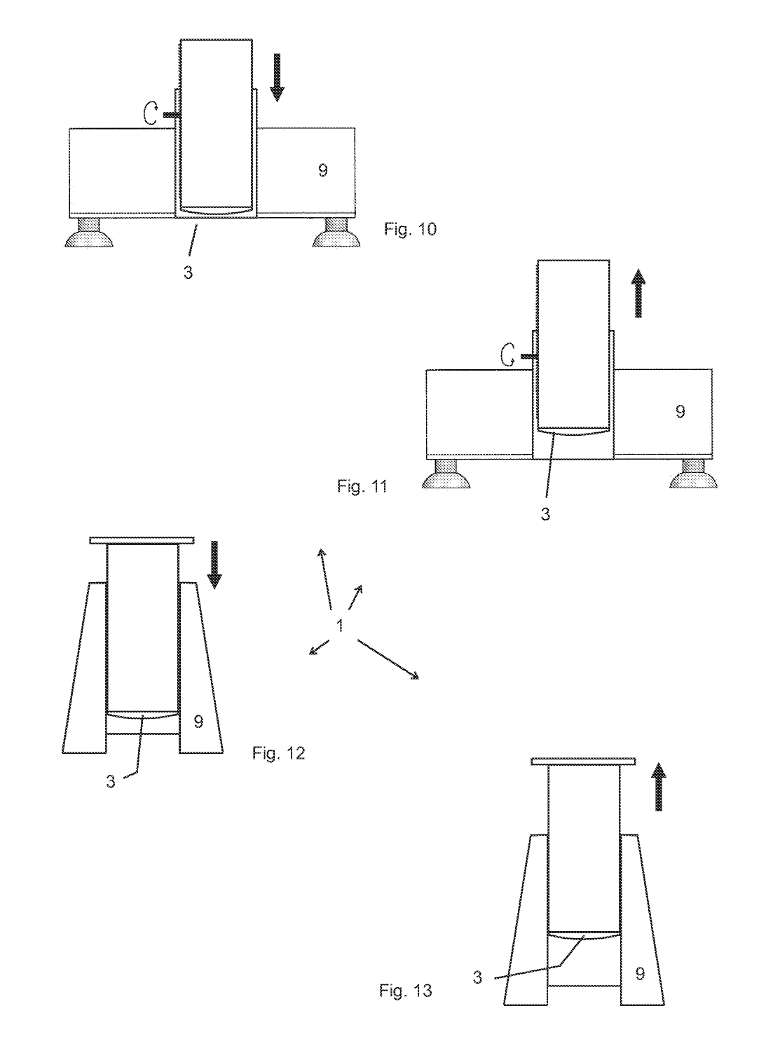

A first non-limiting configurative variation of the equipment 1 presents a mechanism similar to that of the optical microscopes for varying the focus. This mechanism for varying the focus may be any one that varies the distance between two lenses so as to alter the focus. On the one hand, the variation of the focus may either concentrate more brightness and irradiation in a more concentrated manner (imparting a higher average irradiation value per unit of area) or, if used in an opposite manner, may disperse the brightness ion a larger area (distributing the same value of total irradiance over a larger area, which leads to a lower average value of irradiance per unit of area). This spacing may take place by means of a rack-and-pinion mechanism using a screw, for example (see FIGS. 10 and 11), by means of a telescopic system similar to that used on spot-lights that have varying focus (see FIGS. 12 and 13), a system similar to that presented by the “zoom” of a photographic camera, in which one of the lenses performs an angular and linear composed movement in order to alter the focus, or still in any other manner.

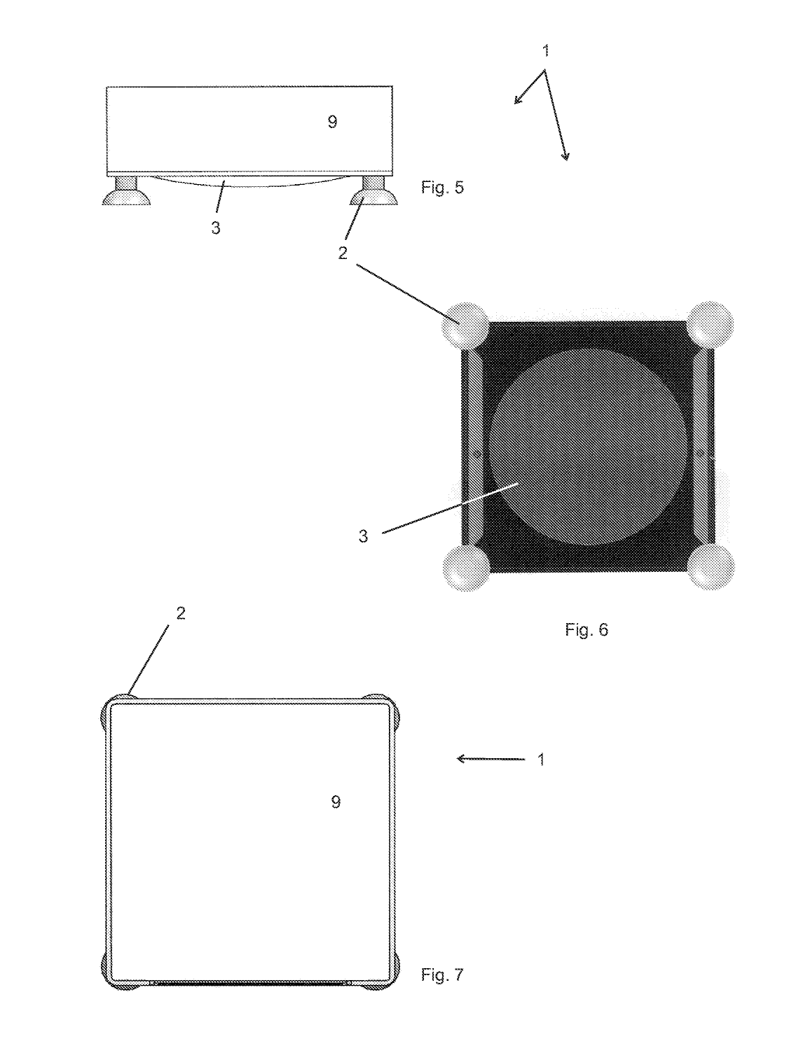

Another constructive variation of the equipment 1 (see FIGS. 4 to 7) has a single additional convex lens 3, foreseen on the body 9, which is supported by a mechanism for varying the focus in the form of one or more feet 3 or base 4, which enables one to vary its height as a whole (see FIG. 4). in this case, the body 9 itself of the equipment 1 rises and lowers with respect to its feet or base, moving away or approaching the single lens 3 with respect to the patient. Evidently, with the lens 3 moving away, the same irradiance/brightness is spread over a larger area A1, reducing the irradiance value. When the body 9 of the equipment 1 is approached to the patient, the same irradiance/brightness is concentrated on a smaller area A2, increasing the irradiance.

Such a solution, together with the progressive proportional control of the irradiance thanks to its microprocessing capability, enables one to maintain the irradiance by microprocessing adjustment, at the same time as the application area increases or decreases. For instance, when treating a 35-week-old patient, one adjusts the focus so that the whole desired portion of his body will be irradiated, and one adjusts the desired irradiance value. After this, the same equipment is used to treat a 50-week-old patient, who is bigger. It is enough to adjust the focus of the lens (lenses) for the larger body area to be illuminated, and the proportional control of irradiance enables the irradiance value emitted by the high-intensity LEDs 5 to be adjusted, so that the same average of the previous patient can be maintained.

Preferably, if there is a radiometer associated to the equipment 1, it is enough to insert it into the illuminated area for this bigger patient, and the irradiance data obtained by the radiometer will be sent to the equipment 1 and processes by the microprocessor, which will then increase automatically the level of radiance according to the treatment protocol. Thus, it is enough for the operator of the apparatus to position the patient on the bed under the equipment 1 and regulate the focus so as to cover the patient as desired. After this, the operator measures the irradiance value with the radiometer, and this data is sent to the equipment 1, which automatically adjusts the value according to the treatment protocol (servo-controlled).

The equipment 1 shown in FIG. 4 has a base 4 with a shape analogous to that of the body 9 and that moves downward to the body, enabling it to move away from the surface on which the base is supported, and vice-versa. In this way, one manages to vary the above-mentioned focus.

The equipment illustrated in FIGS. 5 to 7, in turn, has four individual feet 2, which are telescopic, equally enabling the body to move away from the surface on which it is supported, and vice-versa. In this way, one manages to vary the focus mentioned above.

Moreover, any other solution that enables the lens to move away with the consequent alteration of focus may be used, without the resulting equipment failing to be included in the protection scope of the accompanying claims.

With this solution, it becomes necessary to position the equipment 1 at the end of a movable rod of a pedestal with rotary castors for adjusting the distance between from the patient, which increases the advantage of using it over the items of equipment 100 known at present, as mentioned before.

Thus, the present equipment 1 enables automatic control of the irradiance, with uniform spreading and according to the size of the patient and the area of his body to be treated. None of the existing items of equipment has this capability, which makes the present invention a quite inventive improvement.

If one considers that the efficacy of phototherapy is the product “Area×Irradiance”, and bearing in mind the protocol changes that aim at defining different standards for various types of patients, this is studied now in greater depth, as mentioned before.

As it is known by those skilled in the art, the high-intensity LEDs 5 have estimated useful life of about 50,000 hours and, as this time period is exceeded, the light emitted by it begins to lose the properties that make it so interesting for use in phototherapy, bringing about its exchange. With progressive automatic adjustment of irradiance, coupled with the use of a radiometer, one can compensate automatically the drop in efficiency of the high-intensity LEDs 5, keeping the efficacy of the treatment.

Moreover, the equipment 1 may function as a validator of the phototherapy treatment, because it enables verification of whether it has been carried out with the high-intensity LEDs 5 within or beyond its useful life, verified by means of an incorporated hour-meter for metering the time of treatment or the useful life of the LEDs.

Thus, the present equipment 1 increases or decreases the irradiance emitted by the high-intensity LEDs in an automatic manner, preventing the equipment from functioning in the condition of desired irradiance (pre-established or determined manually), and is capable of keeping the desired irradiance level throughout the phototherapy treatment, since the automatic control corrects possible deviations of the irradiance level resulting from a possible rise in temperature or wear of components and of the sources, etc., without the for manual intervention by an operator/user.

Other great advantage is that the present equipment automatically manages to take into account the type of light emitter, further considering the correction factor. The microprocessor applies at least one correction factor to the irradiance value initially obtained, according to the type of light source selected through a selection means. In this way, the correction is preferably made by means of a computer algorithm to be executed by the microprocessor or microcontroller, with only the operator indicating the type of source of light used.

The functionalities of the equipment 1 having been described in detail, its advantages are summarized hereinafter.

The collimating lens 3 used, with different opacities, makes the irradiance more constant and uniform in its average value, rendering it possible even to reduce the number of high-intensity LEDs 5 used, besides enabling the focal adjustment thereof and enabling the control of the irradiated spot, which facilitates the positioning of the equipment 1 over the patient, and it can be sued on both very small premature patients and bigger patients.

The control of irradiance, which should be carried out after adjusting the irradiated area, cause the treatment to be uniform with the irradiance value being defined for each of the patients.

Taking into consideration the clinical studies that indicate a tendency to reduce irradiances in conformity with the existing protocol levels, which vary according to the intensity of the Kernicterus and other hemolytic diseases, damages to cells, DNA, etc., which are being studied at different degrees of intensity, it is necessary to adjust the irradiated area and the irradiance applied, bringing more advantages to the treatment.

One eliminates the need to use vertically and/or horizontally adjustable supports with wheels that are traditionally troublesome in handling, and chiefly due to the difficulties in coupling the castors to other items of equipment, which are replaced by the adjustable-focus-type system described presently; besides, it is possible to alter the luminous spot without having to use the troublesome supports with adjustable rods or articulates arms—it is possible to adjust the irradiated spot in an unheard-of manner, without repositioning the equipment 1.

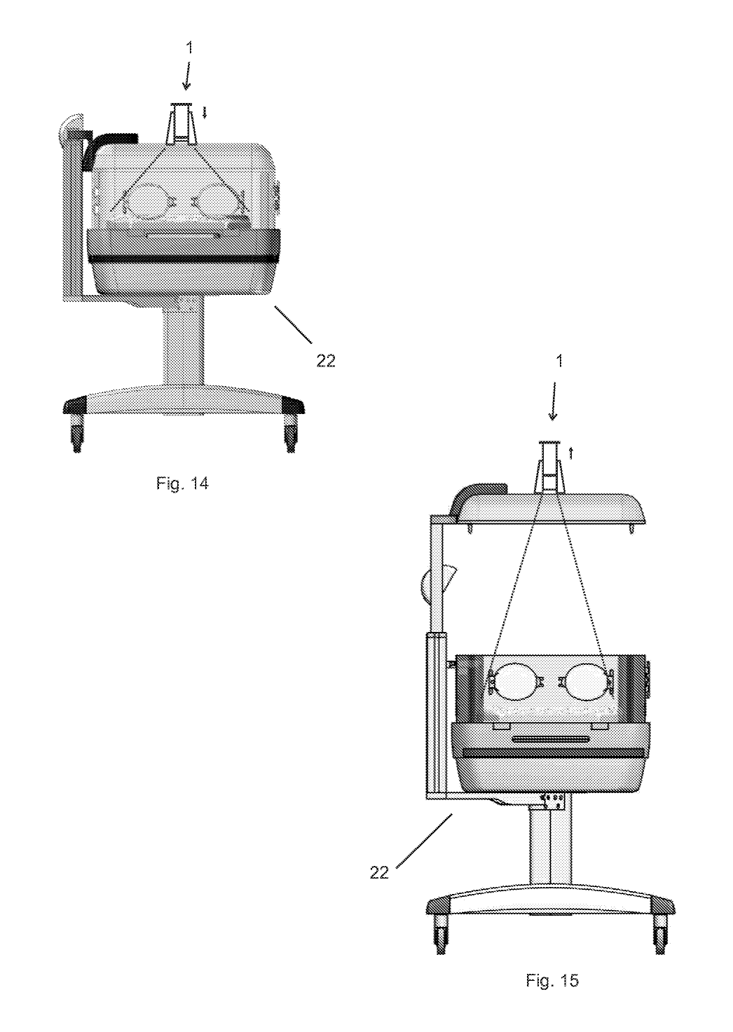

In the event that the equipment 1 is coupled to a hybrid incubator 22, when the dome is raised one does not lose the focus and the irradiance is not impaired; it is possible to adjust the brightness focus on the basis of the height of the bed in conformity with the determined distance (see FIGS. 14 and 15). In this situation, one may conceive the equipment with electric adjustment of the lens 3 in order to dover the whole bed area in a responsive manner, that is to say, so that the equipment itself will adjust the lens focus automatically as the incubator dome is lifted or lowered, servo-controlling the irradiance value with a view to keeping its ideal value for the patient under treatment.

Preferred examples of embodiments having been described, one should understand that the scope of the present invention embraces other possible variations, being limited only by the contents of the accompanying claims, which embrace the possible equivalents.

What is claimed is:

1. A phototherapy treatment device, comprising:
a body comprising at least one source of phototherapeutic light capable of emitting irradiance;
an internal processing element configured to control the emitted radiance for a phototherapy treatment;
one or more collimating lenses positioned adjacent to said at least one source of phototherapeutic light in the direction of the emitted irradiance; and
a focus varying device operatively associated with the one or more collimating lenses.

2. The device according to claim 1, wherein the internal processing element is a microprocessor.

3. The device according to claim 1, wherein the source of phototherapeutic light comprises at least one high-intensity light-emitting diode (LED).

4. The device according to claim 3, wherein the source of phototherapeutic light comprises at least five high-intensity LEDs.

5. The device according to claim 1, wherein the one or more collimating lenses are partially opaque.

6. The device according to claim 5, wherein one or more collimating lenses of the one or more collimating lenses comprises a different opacity from at least one other collimating lens in the one or more collimating lenses.

7. The device according to claim 5, wherein the one or more collimating lenses comprises a single partially opaque collimating lens.

8. The device according to claim 1, wherein the focus varying device comprises a base, wherein the base moves downward and upward with respect to the body to vary the focus.

9. The device according to claim 8, wherein the device does not contain adjustment pedestals.

10. The device according to claim 8, wherein the base comprises a shape analogous to the body.

11. The device according to claim 8, wherein the focus varying device comprises a set of telescopic feet configured to extend from the body.

12. The device according to claim 1, wherein the device enables progressive proportional control of irradiance.

13. The device according to claim 12, wherein the device is servo-controlled.

14. The device according to claim 2 further comprising:
a radiometer associated operatively coupled to the microcessor, the radiometer positioned to measure the emitted irradiance.

15. The device according to claim 14, wherein the radiometer wirelessly communicates with the microprocessor.

16. The device according to claim 1 further comprising a rechargeable battery.

17. The device according to claim 1 further comprising an electromagnetic-wave-transmitting antenna.

18. The device according to claim 17, wherein the electromagnetic-wave-transmitting antenna utilizes Wi-Fi, Bluetooth, or Zigbee.

19. The device according to claim 1, wherein the device is being used as hospital equipment.
